(19) United States
(12) Patent Application Publication
(10) Pub. No.: US 2025/0281026 A1
(43) Pub. Date: Sep. 11, 2025

(54) SEALING DEVICE FOR USE IN ENDOSCOPY

(71) Applicant: Slawomir MARECIK, Northfield, IL (US)

(72) Inventor: Slawomir MARECIK, Northfield, IL (US)

(21) Appl. No.: 19/071,929

(22) Filed: Mar. 6, 2025

Related U.S. Application Data
(60) Provisional application No. 63/561,795, filed on Mar. 6, 2024.

(51) Int. Cl. A61B 1/00

(52) U.S. Cl. CPC A61B 1/00131; A61B 1/00154

(57) ABSTRACT

A sealing device for use in endoscopy includes an elastic body with a leading end, a trailing end, and an inner surface defining a through opening. The outer surface has a smallest diameter in a middle portion and increases toward both ends, forming an hourglass contour that helps anchor within a patient's tissue. The inner surface also has a smallest diameter in its middle portion and broadens toward the ends, permitting a reliable seal around an endoscopic instrument. A slit extends from the leading end toward the middle portion, through the thickness of the body, to allow selective radial deformation during insertion and use. By conforming to various anatomical sizes and maintaining insufflation, this sealing device improves procedural efficiency and patient comfort during colonoscopy or other endoscopic examinations.

### CROSS-REFERENCE TO RELATED APPLICATIONS

This application claims the benefit of U.S. Provisional Application No. 63/561,795, filed Mar. 6, 2024, the entire content of which is hereby incorporated by reference.

### TECHNICAL FIELD

The present invention relates to a sealing device for use in endoscopy, in particular in colonoscopy.

### BACKGROUND

Endoscopic medical procedures allow to visualize and examine interior surfaces of an organ or a cavity within a patient's body. They are performed by means of endoscopes, which are long, flexible tubes with a light source, a camera and various tools on a tip thereof.

Various endoscopic procedures may require insufflation of the cavity within which the endoscope operates, i.e. inflating the cavity with a gas (such as air or carbon dioxide) to provide more workroom. For efficient insufflation, it is preferable for the cavity to be relatively tightly sealed. Therefore, there is a need for appropriate sealing devices that can function as an inlet port to the patient's body and seal the space between the endoscopic tube and the patient's body at the point of entrance of the endoscope to the body.

Sealing may be particularly problematic during colonoscopic procedures, due to differences in sizes of anal canals of various patients.

There is a need to provide an improved sealing device for use in endoscopy, in particular in colonoscopy, to allow efficient sealing of body openings of various sizes.

### SUMMARY OF THE INVENTION

The invention relates to a sealing device for use in endoscopy, having an elastic body with an outer surface and an inner surface defining a through opening extending from a leading end to a trailing end of the elastic body, wherein the outer surface has a round cross-section having a smallest outer diameter in a middle portion, wherein the outer diameter increases from the middle portion towards each of the leading end and the trailing end of the sealing device. The inner surface has a round cross-section having a smallest inner diameter in a middle portion, wherein the inner diameter increases from the middle portion towards each of the leading end and the trailing end of the sealing device, and the body has a slit extending from the leading end towards the middle portion.

In a specific aspect, the invention relates to a sealing device for use in endoscopy, comprising an elastic body having a leading end, a trailing end, an outer surface, and an inner surface defining a through opening extending from the leading end to the trailing end; wherein the outer surface has a round cross-section with a smallest outer diameter at a middle portion, and wherein the outer diameter increases from the middle portion toward each of the leading end and the trailing end; wherein the inner surface has a round cross-section with a smallest inner diameter at the middle portion, and wherein the inner diameter increases from the middle portion toward each of the leading end and the trailing end; and wherein the body has a slit extending between the outer surface and the inner surface from the leading end toward the middle portion.

In use of the sealing device, the outer surface is configured for maintaining a sealing relationship with tissue upon insertion of the sealing device into the tissue for accessing an underlying tissue site. The inner surface is configured for maintaining a sealing relationship with an endoscopic tube at its middle portion.

Since the inner surface has a cross-sectional inner diameter increasing from a middle portion towards each of the leading end and the trailing end, the ends, are relatively flexible and can deform to adapt to the anal canal when the sealing device is installed.

Thereby, the sealing is achieved across a relatively long portion of the anal canal, at the ends of the sealing device, and not only in its most narrow portion, in the middle part of the sealing device.

The slit formed at least at the leading edge facilitates deformation of the sealing device once it is installed in the anal canal so as to more closely adhere to the inner walls of the anal canal, thereby achieving efficient sealing.

The body can be made of an elastic material having Shore A hardness between 20 and 50, preferably 30.

The body can be made of silicone, rubber or a thermoplastic elastomer.

The diameter of the outer surface at the leading end can be smaller than the diameter of the outer surface at the trailing end. This improves usability of the device as it is installed in the anal canal, where the leading portion to be located within the anal canal can be smaller than the portion that remains outside the patient's body. Alternatively, if the device is to be used with a patient having a large diameter of the anal canal, the sealing device can be used in a reverse manner, namely by inserting the larger diameter end into the anal canal.

Alternatively, the diameter of the outer surface at the leading end can be equal to the diameter of the outer surface at the trailing end. This simplifies manufacture of the sealing device and facilitates its use.

The outer surface and the inner surface may each have a shape of a hyperbolic hyperboloid. Such a shape facilitates optimal installation of the sealing device.

The outer surface and the inner surface may each have a cylindrical shape in the middle portion. This increases the sealing properties along the endoscopic tube and increases the coupling between the sealing device and the endoscopic tube.

The thickness of the body can be maximal at the middle portion and can decrease to a minimal thickness towards each of the end portions.

The slit in a side projection can be slanted with respect to the length direction of the sealing device. This facilitates deformation of the end portion of the sealing device when it is inserted into a narrow anal canal.

The slit may extend across the whole length of the sealing device, from its leading end to the trailing end.

The slit can be curved or v-shaped in a side projection, having an apex in the middle portion of the elastic body. This facilitates deformation of the elastic body.

The sealing device can be used in securing transrectal access during endoscopic examination such as colonoscopy, enteroscopy and (recto) sigmoidoscopy.

The device can be used in a method of performing an endoscopic procedure, comprising: providing the sealing device; adapting a diameter of the sealing device at the leading end by flexing the sealing device about the slit so as to conform to a body orifice of a patient; inserting the sealing device into the body orifice of the patient in connection with the endoscopic procedure; positioning an endoscope through the through opening of the sealing device; and insufflating the patient's cavity while maintaining a seal between the sealing device and the patient's tissue.

These and other features, aspects and advantages of the invention will become better understood with reference to the following drawings, descriptions and claims.

### BRIEF DESCRIPTION OF DRAWINGS

Various embodiments are herein described, by way of example only, with reference to the accompanying drawings, wherein:

FIG. 1 shows an overall view of the sealing device;

FIG. 2 shows a side view of a first embodiment of the sealing device;

FIG. 3 shows a side view of the first embodiment of the sealing device with dimensional indicators;

FIG. 4 shows a side view of a second embodiment of the sealing device;

FIG. 5 shows a side view of a third embodiment of the sealing device;

FIG. 6 shows a side view of a fourth embodiment of the sealing device;

FIG. 7 shows a side view of a fifth embodiment of the sealing device;

FIG. 8 shows a side view of a sixth embodiment of the sealing device;

FIG. 9 shows a side view of a seventh embodiment of the sealing device;

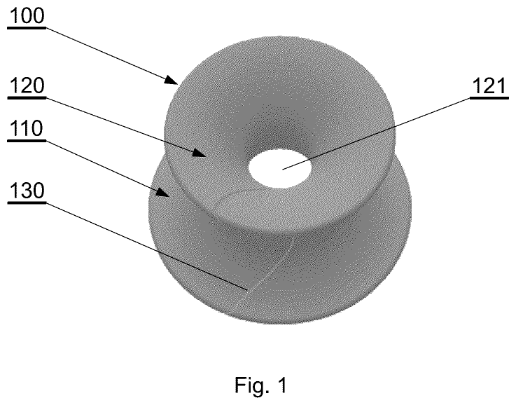

### DETAILED DESCRIPTION OF EMBODIMENTS

The following detailed description is of the best currently contemplated modes of carrying out the invention. The description is not to be taken in a limiting sense, but is made merely for the purpose of illustrating the general principles of the invention.

The sealing device according to the present invention is designed for use in endoscopy and in particular in colonoscopy. It has an elastic body 100 with an outer surface 110 and an inner surface 120. A through opening 121 extends from a leading end 101 to a trailing end 102 of the elastic body 100. The outer surface has a round cross-section that has a smallest outer diameter d_{1c} in a middle portion and that increases from the middle portion towards each of the leading end 101 and the trailing end 102. Similarly, the inner surface 120 has a round cross-section having a smallest inner diameter d2c in a middle portion that increases from the middle portion towards each of the leading end 101 and the trailing end 102. The outer surface 110 and the inner surface 120 may have a cylindrical shape in the middle portion. An exemplary, but non-limiting three-dimensional shape that meet these criteria is a hyperbolic hyperboloid. The body has a slit 130 extending from the leading end 101 towards the middle portion of the elastic body, thereby facilitating deformation of the leading end 101.

The body 100 is made of an elastic material having Shore A hardness between 20 and 50, preferably 30, such as silicone, rubber or a thermoplastic elastomer.

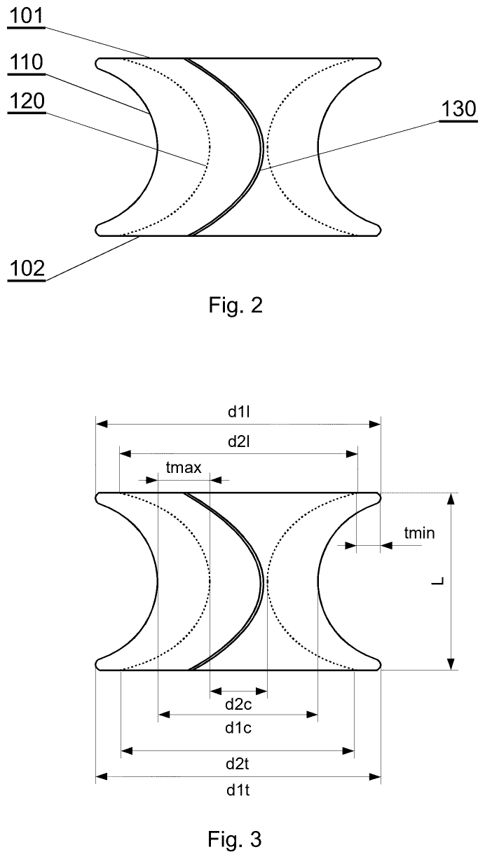

In a first embodiment of the sealing device, as shown in FIG. 2 and FIG. 3 wherein the dotted lines represent the contour of the inner surface 120, the diameter d₁ₗ of the outer surface 110 at the leading end 101 equal to the diameter d₁ₜ of the outer surface 110 at the trailing end 102.

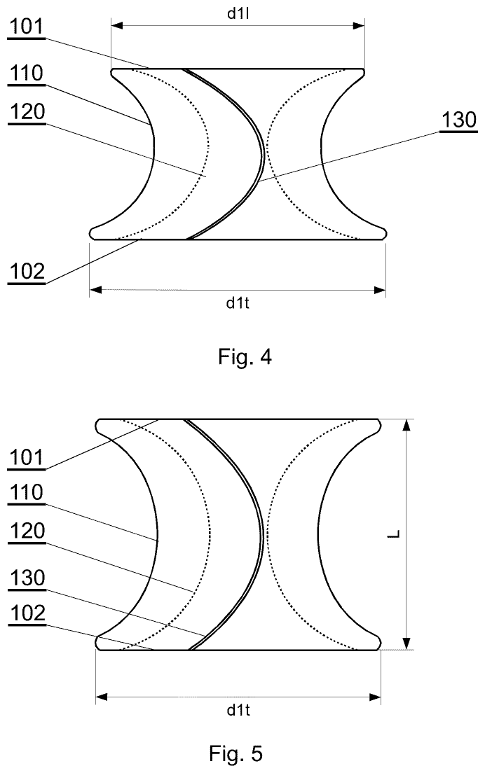

In turn, a second embodiment of the sealing device as shown in FIG. 4, differs from the first embodiment in that the diameter d₁ₗ of the outer surface 110 at the leading end 101 is smaller than the diameter d₁ₜ of the outer surface 110 at the trailing end 102.

The sealing device may have various proportions between the diameter and the length, such as the largest external diameter d₁ₜ may be larger than the length L of the device as shown in most embodiments or smaller than the length L of the device as shown in the third embodiment of FIG. 5.

The thickness of the body 100 is preferably maximal tₘₐₓ at the middle portion and decreases to a minimal thickness tₘᵢₙ towards each of the end portions 101, 102. Preferable, but non-limiting dimensions of the sealing device are:
external diameter d₁ₗ at the leading edge: from 30 to 70 mm, preferably 50 mm
external diameter d_{1c} at the middle portion: from 20 to 50 mm, preferably 30 mm
external diameter d₁ₜ at the trailing edge: from 30 to 70 mm, preferably 55 mm
internal diameter d_{1c} at the middle portion: from 9 to 15 mm, preferably 13 mm
the maximum thickness tₘₐₓ at the middle portion: from 5 to 11 mm, preferably 8.5 mm
the minimum thickness tₘᵢₙ at the end portions can be from 1 to 10 mm, preferably 5 mm.

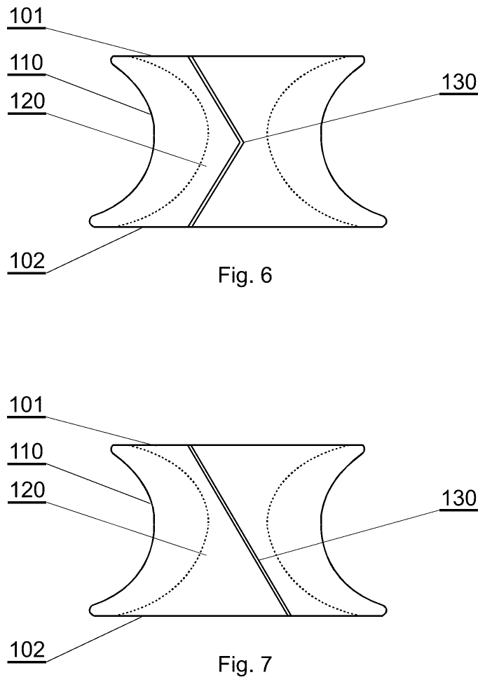

In the embodiments shown in FIGS. 2-5, the slit 130 has a curved shape in a side projection, with an apex in the middle portion of the elastic body 100. However, the slit may have also a V-shaped shape in the side projection, such as shown in the fourth embodiment in FIG. 6, or a straight-line shape, slanted with respect to the length direction of the sealing device, as shown in the fifth embodiment in FIG. 7.

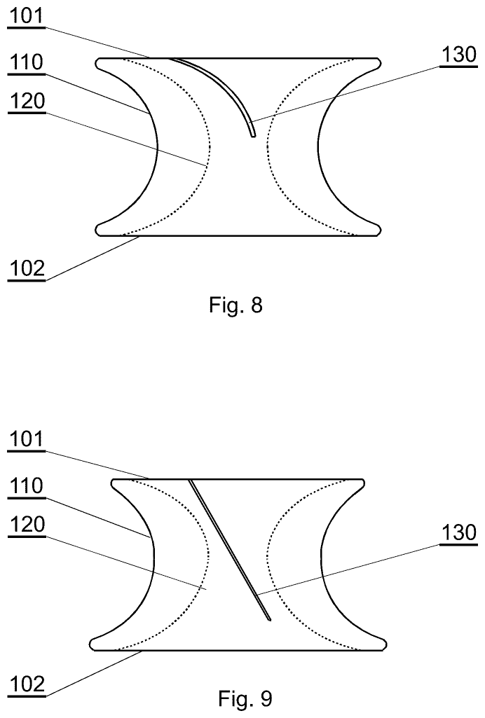

In the embodiments shown in FIGS. 2-7, the slit 130 extends across the whole length of the sealing device, namely from its leading end 101 to the trailing end 102. However, the slit may also terminate before the middle portion of the elastic body, as shown in the sixth embodiment in FIG. 8, or beyond the middle portion of the elastic body, as shown in the seventh embodiment in FIG. 9.

In use, the present sealing device can be employed to secure access for various endoscopic procedures. Referring to the illustrative embodiments described above, the sealing device is inserted into a bodily orifice (such as the anus for colonoscopy, or another orifice suitable for endoscopic procedures). The slit extending from the leading end toward the middle portion enables the leading end to flex radially or partially open, facilitating a snug yet comfortable placement in patient anatomy of varying diameters.

First, a sealing device, as described in the foregoing paragraphs, is selected. The sealing device is configured so that the slit at the leading end can temporarily widen or flex. This adaptation allows the leading end to conform to the shape and size of the patient's body orifice, such as the anal canal during a colonoscopic procedure. Once the device is suitably inserted into the patient's orifice, the elastic body exerts an outward radial pressure, which improves sealing engagement with the surrounding tissue. After or during insertion of the sealing device, an endoscope is advanced through the through opening of the device. The middle portion of the sealing device, having the smallest inner diameter, forms a reliable radial seal around the endoscope shaft. The practitioner then insufflates the patient's cavity with gas, while the device's hourglass geometry and slit-enabled flexibility maintain a fluid-tight or gas-tight seal around both the endoscope and the patient's tissue. This prevents unwanted leakage of insufflation gas and preserves the expanded cavity for efficient visualization or instrument maneuvering.

Although the method is described primarily in connection with colonoscopy via transrectal insertion, the device is equally suitable for other endoscopic procedures such as sigmoidoscopy, enteroscopy, or any procedure requiring sealed access through a body orifice. The diameter ranges, slit geometry, and material elasticity described above can be chosen based on the specific orifice dimensions and procedural requirements.

While the invention has been described with respect to a limited number of embodiments, it will be appreciated that many variations, modifications and other applications of the invention may be made. Therefore, the claimed invention as recited in the claims is not limited to the embodiments described herein.

1. A sealing device for use in endoscopy, comprising:
an elastic body having a leading end, a trailing end, an outer surface, and an inner surface defining a through opening extending from the leading end to the trailing end;
wherein the outer surface has a round cross-section with a smallest outer diameter at a middle portion, and wherein the outer diameter increases from the middle portion toward each of the leading end and the trailing end;
wherein the inner surface has a round cross-section with a smallest inner diameter at the middle portion, and wherein the inner diameter increases from the middle portion toward each of the leading end and the trailing end; and
wherein the body has a slit extending between the outer surface and the inner surface from the leading end toward the middle portion.

2. The sealing device of claim 1, wherein the elastic body is formed of a material having a Shore A hardness between 20 and 50.

3. The sealing device of claim 1, wherein the elastic body is formed of silicone, rubber, or a thermoplastic elastomer.

4. The sealing device of claim 1, wherein a diameter of the outer surface at the leading end is smaller than a diameter of the outer surface at the trailing end.

5. The sealing device of claim 1, wherein a diameter of the outer surface at the leading end is equal to a diameter of the outer surface at the trailing end.

6. The sealing device of claim 1, wherein the outer surface and the inner surface each define a hyperbolic hyperboloid shape.

7. The sealing device of claim 1, wherein the outer surface and the inner surface each have a cylindrical shape at the middle portion.

8. The sealing device of claim 1, wherein a thickness of the elastic body is maximal at the middle portion and decreases toward each of the leading end and the trailing end.

9. The sealing device of claim 1, wherein the slit is slanted with respect to a longitudinal direction of the sealing device in a side projection.

10. The sealing device of claim 1, wherein the slit extends entirely from the leading end to the trailing end.

11. The sealing device of claim 1, wherein the slit is curved or V-shaped in a side projection and has an apex at the middle portion of the elastic body.

12. The sealing device of claim 1, wherein the outer diameter of the middle portion is within a range of 20 millimeters to 50 millimeters.

13. The sealing device of claim 1, wherein the outer diameter of the leading end is within a range of 30 millimeters to 70 millimeters.

14. The sealing device of claim 1, wherein the inner diameter of the middle portion is between 9 millimeters and 15 millimeters.

15. The sealing device of claim 1, wherein the minimum thickness at the leading end and the trailing end is between 1 millimeter and 10 millimeters.

16. The sealing device of claim 1, wherein the slit terminates at the middle portion.

17. The sealing device of claim 1, wherein the slit extends beyond the middle portion and terminates short of the trailing end.

18. The sealing device of claim 1, wherein a maximum outer diameter of the sealing device is greater than an overall length of the device.

19. The sealing device of claim 1, wherein an overall length of the device is greater than a maximum outer diameter of the device.

20. A method of performing an endoscopic procedure, comprising:
providing a sealing device according to claim 1, the sealing device including a slit extending from a leading end toward a middle portion so as to allow radial deformation of the leading end;
adapting a diameter of the sealing device at the leading end by flexing the sealing device about the slit so as to conform to a body orifice of a patient;
inserting the sealing device into the body orifice of the patient in connection with the endoscopic procedure;
positioning an endoscope through the through opening of the sealing device; and
insufflating the patient's cavity while maintaining a seal between the sealing device and the patient's tissue.
